(19) United States
(12) Patent Application Publication
(10) Pub. No.: US 2015/0273062 A1
(43) Pub. Date: Oct. 1, 2015

(54) MAGNESIUM HYDROXIDE CARBONATE AS CARRIER MATERIAL IN ACTIVE INGREDIENT-CONTAINING PREPARATIONS

(71) Applicant: Merck Patent GmbH, Darmstadt (DE)

(72) Inventors: Guenter Moddelmog, Reinheim (DE); Roberto Ognibene, Darmstadt (DE); Thorsten Wedel, Stockstadt/Rhein (DE); Dieter Lubda, Bensheim (DE)

(73) Assignee: Merck Patent GmbH, Darmstadt (DE)

(21) Appl. No.: 14/428,396

(22) PCT Filed: Aug. 19, 2013
(86) PCT No.: PCT/EP2013/002490

§ 371 Date: Mar. 16, 2015

(30) Foreign Application Priority Data
Sep. 18, 2012 (EP) 12006551.1
Feb. 13, 2013 (EP) 13000730.5

(51) Int. Cl. A61K 47/02; A61K 31/375; A61K 31/525; A61K 9/16

(52) U.S. Cl. CPC A61K 47/02; A61K 9/1611; A61K 31/375; A61K 31/525

(57) ABSTRACT

The present invention relates to solid formulations which contain at least one porous carrier and one or more functional substances in a stable mixture, and to the use thereof.

The present invention relates to solid formulations which comprise at least one porous carrier and one or more functional substances, and to the use thereof.

### PRIOR ART

Active ingredients (APIs) for use in pharmaceutical administration forms must on the one hand have processing properties which are usable for pharmaceutical practice in order that the active ingredient is suitable at all for pharmaceutical formulation to give the final medicinal form. On the other hand, even active components which are problematic to process can be converted into patient-suitable formulations, such as, for example, powders, granules, capsules or tablets, through a suitable choice of an inert medicament carrier. The carriers used for such purposes must have particular physical or chemical properties, depending on the problem and active ingredient, in order to compensate for the processing deficiencies of the API.

The formulation scientist is faced with a particular challenge in cases where the administration advantageously takes place in powder form, but the active ingredient is particularly finely divided and/or has to be employed in a low dose. In such cases, it is desirable to formulate the active ingredient together with a suitable carrier. Owing to their biological properties, it is desirable to dilute, in particular, highly active, low-solubility active ingredients with on a pulverulent carrier in such a way that the active ingredient can be made available in constant dose on administration. In a first approach, it would be obvious simply to mix the active ingredient with a carrier material which can conventionally be employed for the production of tablets in order to be able to present a dispensable amount of powder. However, it is problematic in this connection that the carrier material and the pulverulent active ingredient must not separate out for a uniform dose over the long term. This plays a role, in particular, during storage and handling of the powder formulation if the active ingredient is in the form of significantly smaller particles than the carrier material. In such mixtures, the active ingredient in the form of relatively small particles has the tendency to trickle downwards, while the relatively large carrier particles apparently migrate upwards and remain there. In order to prevent this separation effect, it has been attempted in the recent past to prepare corresponding formulations by granulation in the presence of suitable solvents. However, this is associated with additional process steps and increased energy consumption. In addition, this process is also unsuitable for active ingredients which have instabilities, due to dissolution steps and drying processes necessary for their preparation and further processing.

A very wide variety of carrier and filler materials are known per se for the preparation of solid, active ingredient-containing formulations.

Use is frequently made of microcrystalline cellulose, which is prepared from wood pulp or crude cellulose by warming with mineral acids and is subsequently converted into a finely particulate form by mechanical comminution of the cellulose aggregates. This cellulose exhibits plastic flow during compression and is counted amongst the viscoelastic substances. Microcrystalline cellulose is used as filler and dry binder in direct tableting.

Another plastic carrier and filler material is starch, preferably water-soluble, directly compressible starch, which has both plastic and also elastic (viscoelastic) deformation behaviour. Corresponding starch products are used in direct tableting as filler and disintegrant.

Various forms of lactose are used particularly frequently as carriers for mixtures, granules, hard gelatine capsules and tablets. Lactose can be used both as monohydrate and also in anhydrous form. In addition, it occurs in various modifications, in some cases with amorphous contents, depending on the preparation process. Thus, for example, spray-dried lactose, which has a high amorphous content, can be employed as directly compressible tableting assistant. Lactose variants are offered by various suppliers in a very wide variety of particle sizes and particle morphologies for a very wide variety of applications.

In the recent past, the sugar alcohols, such as mannitol, sorbitol, xylitol, have additionally become more and more important as tableting assistants with the function as carrier and filler material. As spray-dried, optionally granulated products, they are directly compressible.

However, it is not desired to use organic carrier and filler materials for every formulation, owing to undesired interactions with an active ingredient or in the case of intolerance by the user.

As a replacement, it has therefore been attempted to use inorganic salts for tableting, preferably those which are well tolerated and themselves do not exhibit any side effects when employed in the usual amounts.

Thus, calcium hydrogen phosphate dihydrate is marketed as tableting assistant under the trade name Di-Cafos®. It is prepared by reaction of calcium hydroxide with phosphoric acid at temperatures below 40° C. The monoclinic dihydrate is formed, which occurs in nature as brushite (Gmelins Handbuch der Anorganischen Chemie [Gmelin's Handbook of Inorganic Chemistry], 1961, 8th Edn. Calcium, Part B, 27, publisher Deutsche Chemische Gesellschaft, Verl. Chemie GmbH, Berlin, 321-329). It exhibits a brittle-fracture deformation behaviour which is substantially independent of the pressing rate (Rees, J. E. and P., J. Rue; “Time-dependent deformation of some direct compression excipients”, J. of Pharmacy and Pharmacology 30(10): 601-7. (1978)). The primary particles have low elasticity and high hardness. Di-Cafos® is used as filler and dry binder in direct tableting, as flow regulator in capsule recipes and as abrasive component in toothpastes.

Calcium hydrogen phosphate containing no water of crystallisation is marketed as Fujicalin® for the production of tablets. Calcium hydrogen phosphate is produced industrially by reaction of calcium hydroxide with phosphoric acid at temperatures above 75° C. (Toy A. D. F., Walsh E. N. in: “Phosphorus chemistry in everyday living”, 2nd Edition American Chemical Society, Washington, D.C. (1987)). In tableting, calcium hydrogen phosphate containing no water of crystallisation is usually used as filler and binder, as Ca²⁺ supplier in mineral preparations or as abrasive in dental care compositions. In the synthetic preparation of Fujicalin®, crystal growth is limited (Takami K., Machimura; H., Takado K.; Inagaki M.; Kawashima Y.; in “Novel preparation of free flowing spherically granulated dibasic calcium phosphate anhydrous for direct tabletting”, Chem. Pharm. Bull., 44 (4), 868-870 (1996)) and the size of the crystallites is thus reduced compared with other preparation processes. This is followed by granulation by spray drying (Takado K.; Murakami T.; Japanese patent application Kokai No. 298505 (1994); Takado K.; Murakami T.; Japanese patent application Kokai No. 118005 (1995)), giving virtually spherical, extremely porous particles. This product has a specific surface area of 27 m²/g, a factor of 90 greater than Di-Cafos®.

Inorganic salts which are of interest for the production of tablets are, in particular, carbonates, which can be dissolved easily in water in the presence of acidic substances, enabling the active ingredient to be made available in a fizzy drink. Effervescent tablets are usually produced using sodium carbonate, sodium hydrogencarbonate, calcium carbonate or corresponding potassium carbonates, and citric acid, tartaric acid or also ascorbic acid as acid components.

Under certain conditions, however, it is desirable to be able to provide corresponding tablets in which inorganic salts are used which contain magnesium salts instead of the calcium, sodium or potassium salts as filler and carrier material. This applies not only to the effervescent formulations mentioned, but also to other mixtures, granules or conventional tablets.

However, conventional pulverulent magnesium hydroxide carbonate, owing to its poor flow properties and owing to the lack of compressibility, cannot be employed as carrier or tableted directly without special additives or special pretreatment. This is basic magnesium hydroxide carbonate having the chemical composition 4MgCO₃×Mg(OH)₂×5H₂O. It is only formed from aqueous solution if the latter contains a large excess of carbonic acid. Magnesium carbonate is able to crystallise with 5, 3 and 1 mol of water of crystallisation and is gradually decomposed to basic magnesium carbonate on boiling with water. Corresponding processes for the preparation have been known for a long time.

### Object

The present invention is based on the object of providing a pulverulent carrier material, optionally also in a readily table form, which allows, in a simple, inexpensive manner, the preparation of solid dosage forms in which the active ingredients are distributed as homogeneously as possible and are protected against separation tendencies.

It is furthermore the object of the present invention to provide corresponding formulations in solid form in which the active ingredient, which is optionally in a very low dose or in very finely particulate (micronised) form, is homogeneously distributed. In addition, it is an object of the present invention to provide a directly compressible, active ingredient-containing product having good flowability which allows the preparation of pharmaceutical administration forms having a homogeneous active-ingredient distribution.

### Achievement of the Object

In accordance with the present invention, the object is achieved, surprisingly, by solid formulations which are characterised in that they
a) comprise at least one porous carrier which consists of magnesium hydroxide carbonate, and
b) comprises one or more functional substances.

Corresponding formulations comprise ordered mixtures consisting of 50 to 99.9% by weight of magnesium hydroxide carbonate and 50 to 0.1% by weight of at least one micronised functional component. The magnesium hydroxide carbonate present is preferably a material having a BET surface area in the range 25 to 70 m²/g, preferably greater than 44 m²/g, and a bulk density in the range from 0.40 to 0.60 g/ml, and a tapped density in the range from 0.50 to 0.80 g/ml. Formulations in which a directly compressible magnesium hydroxide carbonate having a particle diameter (laser, D₅₀) in the range between 10 and 60 μm, preferably between 20 and 60 μm, is present and at least one functional component in the form of a micronised substance having a particle size (laser, D₅₀) of 1-20 μm, in particular of 1-10 μm, have surprisingly good properties.

In accordance with the invention, corresponding formulations may comprise at least one functional component from the area of pharmaceutical active ingredients, diagnostic agents, food supplements, cosmetics, herbicides, fungicides, reagents, dyes, dietary minerals or catalysts, as well as enzymes or microorganisms.

These formulations are surprisingly ordered mixtures which consist of 50 to 99.9% by weight of magnesium hydroxide carbonate, and which are distinguished by pronounced homogeneity and stability, even under mechanical load. In accordance with the invention, these formulations may, apart from at least one functional component, comprise active ingredients and assistants selected from the group flow improvers, binders, lubricants, sweeteners and polymers. Unexpectedly, these mixtures can be formulated as powder or tablet. The ordered mixture is stable in the long term as powder and retains its homogeneous active-ingredient distribution even after mechanical loading, such as, for example, by transport or in requisite further processing steps, even if the pharmaceutical active ingredient is present therein in a low dose.

The formulations according to the invention are distinguished by the fact that the porous magnesium hydroxide carbonate present as carrier form, together with one or more functional substances, a stable ordered mixture having particularly good homogeneity which have particularly low separation tendencies.

In accordance with the present invention, the present object is also achieved by the use of the formulations described for the preparation of mixtures in solid, semi-solid and liquid form which are used, for example, for the production of active ingredient-containing tablets, capsules, powders, ointments, creams, suspensions, dispersions. In accordance with the invention, they can also advantageously be used for the preparation of pharmaceutical formulations for oral or dermal administration. The formulations are likewise highly suitable for the preparation of cosmetic, agricultural and industrial formulations or of food preparations and formulations for food supplementation.

The object according to the invention is also achieved, in particular, by a process for the preparation of the formulations described in which at least one porous carrier, consisting of magnesium hydroxide carbonate, and at least one functional substance in the form of a micronised powder are mixed intensively with one another in a mixer selected from the group tumble mixers, screw cone mixers, compulsory mixers, stirred mixers, high-speed mixers and fluidised-bed mixers.

### DETAILED DESCRIPTION OF THE INVENTION

In the production of tablets, various problems arise which have to be solved by the formulation scientist. On the one hand, the various starting materials introduced have to be shaped with one another to give a stable tablet body. On the other hand, all tablets must contain the active ingredient(s) in the same concentration in all cases. However, that is not all. The active ingredient must also be uniformly distributed in each individual tablet, so that the user, when he divides the tablet, finds the same active ingredient concentration in each part of the tablet and is able to take an accurate dose. Depending on the physical properties of the active ingredient or active ingredients which are to be formulated as tablets, various requirements arise therefrom, in particular if tablets with a low dosage are to be formulated.
1. If, for example, the active ingredient is in liquid form, for example as oil, dissolved in aqueous or organic solvents or as dispersion or emulsion, it must firstly, before its use in a solid administration form, be converted into a powder which can be processed further.
2. If the active ingredient is to be used in a very low dose, particular measures must be taken which enable uniform distribution in the solid pharmaceutical administration form to be guaranteed. A corresponding situation applies if the active ingredient is present in such a small particle size that it cannot be mixed with the other constituents of the formulation in a sufficiently stable manner for further tableting. Thus, some medicaments tend towards separation owing to their particle size and particle morphology.
3. In addition, electrostatic phenomena, which can cause inadequately uniform active-ingredient distribution, are also problematic.

In general, corresponding problems can be solved by applying the problematic medicament to a porous carrier before its further processing to give the tablet. This can be carried out in various ways. This is usually carried out in an additional granulation step.

The magnesium hydroxide carbonate described in WO 2011/095269 is distinguished by a special particle morphology, combined with a particularly large BET surface area and a high pore volume.

The magnesium hydroxide carbonate characterised in this way is, owing to its porous structure, readily soluble in an acidic and aqueous environment, such as gastric juice, and liberates CO₂ gas. Depending on the size of a tablet produced therefrom, this magnesium hydroxide carbonate can be employed as carrier material or filler which disintegrate rapidly in the mouth on administration or for the production of active ingredient-containing fizzy drinks.

Experiments have now shown that this porous magnesium hydroxide carbonate is able to bind large amounts of finely particulate pharmaceutical active ingredients.

Surprisingly, the preparation of suitable dosage forms which consist of a predominant proportion of porous magnesium hydroxide carbonate as carrier succeeds in the absence of solvents by simple intensive mixing if the low-solubility active ingredient is in the form of ultrafine powder.

The particular particle properties result in the very fine particulate active ingredients being bonded to the surface of the magnesium hydroxide carbonate particles due to adsorptive interactions merely through intensive mixing and separation thus being prevented, so that the uniform distribution of medicament in the tableted, but in particular also in the pulverulent administration form can be ensured.

Further experiments with liquid active ingredients, optionally in the form of oil, have shown that these can be applied to the magnesium hydroxide carbonate particles by strong adsorption to the surface as such, but also in a dissolved liquid preparation, and can thus be converted into flowable powders which, if desired, can be compressed to give tablets.

The intensive mixing mentioned of the functional component or components with the relatively coarse, porous magnesium hydroxide carbonate particles gives a so-called stable “ordered mixture” of porous magnesium hydroxide carbonate as carrier and at least one functional component. This means that the component present in very dilute form in the mixture can be dispensed uniformly; so that under these conditions variations in the weight of the formulation result in smaller variations in the dose than if the functional component were in undiluted form. This effect is of considerable importance, in particular, for single-dose pharmaceutical administration forms, such as, for example: in the filling of sachets with powders or also in the filling of cavities of tableting machines with the mixture to be tableted.

Dosage forms are taken to mean all forms which are suitable for use as medicaments, in particular for oral administration, and food supplements, but also cosmetics, plant treatment agents, such as herbicides or fungicides, reagents, diagnostic agents and feeds and also as dyes, dietary minerals or catalysts. These include, for example, tablets of any shape, pellets or granules and powder mixtures.

Due to the special properties of the magnesium hydroxide carbonate described in WO 2011/095669, the formulation scientist in the pharmaceutical industry and in the foods industry or in other areas is given the possibility of bringing even active ingredients or materials which are problematic in pharmaceutical formulation terms into a form which can be processed further. Since the magnesium hydroxide carbonate used is a substance which is listed in all pharmacopoeias, there are also no additional requirements to be met regarding registration of the filler and carrier material.

Although the magnesium hydroxide carbonate employed in accordance with the invention is directly compressible, adjuvants may be present in accordance with the invention in the solid formulations comprising active ingredient, besides the active ingredient and the porous magnesium hydroxide carbonate as excipient.

These may be, inter alia, flavour improvers, tableting assistants, such as glidants and lubricants, and the like. Possible additives are, for example, thermoplastic polymers, lipids, sugar alcohols, sugar alcohol derivatives, solubilisers, glidants and lubricants and others.

Suitable thermoplastic polymers are, for example, polyvinyl pyrrolidone (PVP), copolymers of N-vinylpyrrolidone and vinyl acetate or vinyl propionate, copolymers of vinyl acetate and crotonic acid, partially hydrolysed polyvinyl acetate, polyvinyl alcohol, polyhydroxyalkyl acrylates, polyhydroxyalkyl methacrylates, polyacrylates and polymethacrylates (Eudragit products), copolymers of methyl methacrylate and acrylic acid, polyethylene glycols, alkylcelluloses, in particular methylcellulose and ethylcellulose, hydroxyalkylcelluloses, in particular hydroxypropylcellulose (HPC), hydroxyalkylalkylcelluloses, in particular hydroxypropylmethylcellulose (HPMC), cellulose esters, such as cellulose phthalates, in particular cellulose acetate phthalate, hydroxypropylmethylcellulose phthalate and hydroxypropylmethylcellulose acetate succinate (HPMCAS). Thermoplastic polymers of this type are known to the person skilled in the art. He will be able to choose between the thermoplastic polymers which are commercially available for this purpose, depending on the desired properties of the tablets to be produced.

However, low-molecular-weight substances may also be present as additional excipients and fillers in the formulations comprising active ingredient. These can be sugars, such as sucrose, glucose, maltose, xylose, fructose, ribose, arabinose, galactose, trehalose, but also sugar alcohols. Suitable sugar alcohols are sorbitol, xylitol, mannitol, maltitol; a suitable sugar alcohol derivative is also isomaltitol. These additives may be commercially available in various grades under various trade names.

Suitable lipids are fatty acids, such as stearic acid; fatty alcohols, such as cetyl or stearyl alcohol; fats, such as animal or vegetable fats; waxes, such as carnauba wax; or mono- and/or diglycerides or phosphatides, in particular lecithin. The fats preferably have a melting point of at least 50° C. Preference is given to triglycerides of the C₁₂-, C₁₄-, C₁₆- and C₁₈-fatty acids.

In addition, conventional pharmaceutical-formulation adjuvants, whose total amount can be up to 20% by weight, preferably less than 10% by weight, in particular less than 5% by weight, based on the dosage form, can also be used. These include:

diluents or fillers, such as lactose, cellulose, silicates or silicic acid; lubricants, such as magnesium stearate and calcium stearate, sodium stearyl fumarate;
plasticisers;
dyes, such as azo dyes, organic or inorganic pigments or dyes of natural origin; stabilisers, such as antioxidants, light stabilisers, hydroperoxide destroyers, free-radical scavengers, preservatives and stabilisers against microbial infestation;
aromas and fragrances;
anticaking agents;
disintegration-promoting adjuvants (disintegrants)
and retardation agents.

Active ingredients in the sense of the invention are taken to mean all substances having a desired physiological action on the human or animal body or plants. They are, in particular, active pharmaceutical ingredients. The amount of active ingredient per dose can vary within broad limits. It is generally selected so that it is sufficient in order to achieve the desired action. Combinations of active ingredients can also be employed. Active ingredients in the sense of the invention are also vitamins and dietary minerals. The vitamins include the vitamins from group A, group B, which, besides B₁, B₂, B₆ and B₁₂, are also taken to mean in a broader sense nicotinic acid and nicotinamide, and also biotin, folic acid, but also compounds having vitamin-like properties, such as, for example, adenine, choline, pantothenic acid, adenylic acid, orotic acid, pangamic acid, carnitine, p-aminobenzoic acid, myo-inositol and lipoic acid, as well as vitamin C, vitamins from group D, group E, group K. Active ingredients in the sense of the invention also include peptide therapeutic agents and proteins.

In accordance with the invention, the magnesium hydroxide carbonate described in WO 2011/095669 can be employed, for example, for the processing of the following active ingredients in a suitable process:

acebutolol, acetylcysteine, acetylsalicylic acid, aciclovir, albrazolam, alfacalcidol, allantoin, allopurinol, ambroxol, a-mikacin, amiloride, aminoacetic acid, amiodarone, amitriptyline, amlodipine, amoxicillin, ampicillin, ascorbic acid, aspartame, astemizole, atenolol, beclomethasone, benserazide, benzalkonium hydrochloride, benzocaine, benzoic acid, betamethasone, bezafibrate, biperiden, bisoprolol, bromazepam, bromhexine, bromocriptine, budesonide, bufexamac, buflomedil, buspirone, caffeine, camphor, captopril, carbamazepine, carbidopa, carboplatin, cefachlor, cefalexin, cefatroxil, cefazolin, cefixime, cefotaxime, ceftazidime, ceftriaxone, cefuroxime, celedilin, chloramphenicol, chlorhexidine, chlor-pheniramine, chlortalidone, choline, cyclosporin, cilastatin, cimetidine, ciprofloxacin, cisapride, cisplatin, clarithromycin, clavulaeic acid, clomibramine, clonazepam, clonidine, clotrimazole, codeine, cholestyramine, cromoglycinic acid, cyanocobalamine, cyproterone, desogestrel, dexamethasone, dexpanthenol, dextromethorphan, dextropropoxyphene, diazepam, diclofenac, digoxin, dihydrocodeine, dihydroergotamine, dihydroergotoxine, diltiazem, diphenhydramine, dipyridamole, dipyrone, disopyramide, domperidone, dopamine, doxycycline, enalapril, ephedrine, epinephrine, ergocalciferol, ergotamine, erythromycin, estradiol, ethinylestradiol, etoposide, eucalyptus globulus, famotidine, felodipine, fenofibrate, fenofibric acid, fenoterol, fentanyl, flavin mononucleotide, fluconazole, flunarizine, fluorouracil, fluoxetine, flurbiprofen, furosemide, gallopamil, gemfibrozil, gentamicin, gingko biloba, glibenclamide, glipizide, clozapine, glycyrrhiza glabra, griseofulvin, guaifenesin, haloperidol, heparin, hyaluronic acid, hydrochlorothiazide, hydrocodone, hydrocortisone, hydromorphone, ipratropium hydroxide, ibuprofen, imipenem, indomethacin, insulin, iohexol, iopamidol, isosorbide dinitrate, isosorbide mononitrate, isotretinoin, ketotifen, ketoconazole, ketoprofen, ketorolac, labatalone, lactulose, lecithin, levocarnitine, levodopa, levoglutamide, levonorgestrel, levothyroxine, lidocaine, lipase, lipramine, lisinopril, loperamide, lorazepam, lovastatin, medroxyprogesterone, menthol, methotrexate, methyldopa, methylprednisolone, metoclopramide, metoprolol, miconazole, midazolam, minocycline, minoxidil, misoprostol, morphine, multivitamin mixtures or combinations and mineral salts, N-methylephedrine, naftidrofuryl, naproxen, neomycin, nicardipine, nicergoline, nicotinamide, nicotine, nicotinic acid, nifedipine, nimodipine, nitrazepam, nitrendipine, nizatidine, norethisterone, norfloxacin, norgestrel, nortriptyline, nystatin, ofloxacin, omeprazole, ondansetron, pancreatin, panthenol, pantothenic acid, paracetamol, penicillin G, penicillin V, phenobarbital, phenoxifylline, phenoxymethylpenicillin, phenylephrine, phenylpropanolamine, phenytoin, piroxicam, polymyxin B, povidone iodine, pravastatin, prazepam, prazosin, prednisolone, prednisone, promocriptine, propafenone, propranolol, proxyphylline, pseudoephedrine, pyridoxine, quinidine, ramipril, ranitidine, reserpine, retinol, riboflavin, rifampicin, rutoside, saccharine, salbutamol, salcatonin, salicylic acid, simvastatin, somatropin, sotalol, spironolactone, sucralfate, sulbactam, sulfamethoxazole, sulfasalazine, sulpiride, tamoxifen, tegafur, teprenone, terazosin, terbutaline, terfenadine, tetracycline, theophylline, thiamine, ticlopidine, timolol, tranexamic acid, tretinoin, triamcinolone acetonide, triamterene, trimethoprim, troxerutin, uracil, valproic acid, vancomycin, verapamil, vitamin E, volic acid, zidovudine.

In addition, other finely particulate active ingredients which are difficult to administer in accurate doses can also be incorporated into a formulation using magnesium hydroxide carbonate and, if desired, tableted.

For the preparation of solid dosage forms, the carrier and the active ingredients are mixed intensively with one another in a corresponding mixing ratio, preferably in a suitable mixer. The active ingredients are, if they are not already in the form of superfine powder, ground to give a very finely divided powder before the mixing, i.e. the active ingredient is micronised and subsequently has average particle sizes of a few microns, or in the nanometre region. The active ingredients are preferably used as functional components in the form of micronised substances having an average particle size (laser, D₅₀) in the range from 1 to 20 μm, preferably in the range from 1 to 10 μm.

The magnesium hydroxide carbonate which can be employed in accordance with the invention is a porous material having a BET surface area in the range from 25 to 70 m²/g, preferably greater than 44 m²/g, particularly preferably greater than 50 m²/g, and a bulk density in the range from 0.40 to 0.60 g/ml, and a tapped density in the range from 0.50 to 0.80 g/ml, which can be obtained as described in WO 2011/095669.

Depending on the physical properties of the active-ingredient powder, pulverulent magnesium hydroxide carbonate as carrier and the finely divided active ingredient are initially introduced in a suitable mixing ratio and mixed intensively with one another. However, it is also possible to meter the active ingredient in little by little during the mixing in order in this way to achieve a uniform distribution of the active ingredient on the carrier. The mixing of the two components here can be carried out in equipment which is known to the person skilled in the art for this purpose. The mixing is preferably carried out under gentle conditions in a tumble mixer; screw cone mixer, compulsory mixer, high-speed mixer, propeller mixer or in a fluidised-bed mixer. These also allow liquid active ingredients to be mixed uniformly with the solid carrier material.

For the preparation of the desired “active ingredient/carrier adduct”, 50 to 99.9% by weight of magnesium hydroxide carbonate and 50 to 0.1% by weight of a micronised functional component or a liquid functional component, based on the total amount, are initially introduced and mixed with one another in a suitable manner. The directly compressible, porous magnesium hydroxide carbonate used for this purpose preferably has particle diameters (laser, D₅₀) in the range between 10 and 60 μm, particularly preferably between 20 and 60 μm.

The intensive mixing of the functional components gives a mixture which is distinguished, even under mechanical load, by excellent stability of the mixture and pronounced homogeneity of the distribution of the active ingredient on the carrier material.

The special particle morphology of the carrier together with a high BET surface area and a high pore volume result in strong physical adsorption of the finely particulate active-ingredient particles on the carrier surface. So-called ordered mixtures form. In this way, an active ingredient with poor miscibility can be converted into a homogeneous preparation which has virtually no tendency towards separation of the individual components, even under mechanical load. This improves the dosage accuracy of the active ingredient (content uniformity) in individual doses taken from such mixtures.

The active ingredient-containing powder according to the invention, which essentially consists of magnesium hydroxide carbonate as carrier material and the selected active ingredient, has, owing to its porosity, very good tableting properties and has particle diameters (laser, D₅₀) in the range between 10 and 60 μm, preferably between 20 and 60 μm.

The advantage of the preparations provided by the present invention lies in the fact that finely particulate or low-dose active ingredients are homogeneously distributed bonded to a carrier, enabling the preparation of low-dosage preparations which would tend towards separation under conventional conditions.

The mixture according to the invention is advantageously a product which is distinguished, even under mechanical load, by pronounced homogeneity and stability of the mixture. Besides the functional component and the magnesium hydroxide carbonate employed as carrier, it may comprise active ingredients and assistants selected from the group flow improvers, binders, lubricants, sweeteners and polymers.

It is particularly advantageous under the given conditions that the active ingredient, which is optionally in the form of a pure substance in the form of an oil, can be made available as low-dosage powder by the bonding to the porous, pulverulent magnesium hydroxide carbonate. Owing to the porous properties, this powder can, if desired, be tableted directly, giving tablets in which the active ingredient is homogeneously distributed Like in the active ingredient-containing powder, the pharmaceutical active ingredient is in a low dose in the tablet produced, the active ingredient-containing mixture is in solid form both in the tablet and also in the active ingredient-containing powder. The active ingredient-containing powder can in turn advantageously be used for the production of active ingredient-containing tablets, capsules, powders, ointments, creams, suspensions, dispersions, in particular for the preparation of pharmaceutical formulations for oral or dermal administration or of pharmaceutical, cosmetic, agricultural and industrial formulations, food preparations and formulations for food supplementation.

The present description enables the person skilled in the art to apply the invention comprehensively. Even without further comments, it is therefore assumed that a person skilled in the art will be able to utilise the above description in the broadest scope.

If anything is unclear, it goes without saying that the publications and patent literature cited should be consulted. Accordingly, these documents are regarded as part of the disclosure content of the present description. This applies, in particular, to the disclosure content of the application WO 2011/095269, in which the preparation of the magnesium hydroxide carbonate used is described and which is thus part of the disclosure content of the present invention.

For better understanding of the invention and in order to illustrate it, various examples are given below which are within the scope of protection of the present invention. These examples also serve to illustrate possible variants. Owing to the general validity of the inventive principle described, however, the examples are not suitable for reducing the scope of protection of the present application to these alone.

Furthermore, it goes without saying to the person skilled in the art that, both in the examples given and also in the remainder of the description, the component amounts present in the compositions always only add up to 100% by weight or mol-%, based on the composition as a whole, and cannot exceed this, even if higher values could arise from the per cent ranges indicated. Unless indicated otherwise, % data are thus regarded as % by weight or mol-%, with the exception of ratios, which are reproduced in volume data.

The temperatures given in the examples and the description as well as in the claims are always in ° C.

### EXAMPLES

In order to carry out the following examples, the following materials, equipment and measurement methods were used:

### Methods:

1. Bulk density: in accordance with DIN EN ISO 60: 1999 (German version)
   quoted in “g/ml”
2. Tapped density: in accordance with DIN EN ISO 787-11: 1995 (German version)
   quoted in “g/ml”
3. Surface area determined in accordance with BET: evaluation and procedure in accordance with the literature “BET Surface Area by Nitrogen Absorption” by S. Brunauer et al. (Journal of American Chemical Society, 60, 9, 1983), instrument: ASAP 2420, Micromeritics Instrument Corporation (USA); nitrogen; sample weight: about 3.0000 g+/−5%; heating: 50° C. (5 h); heating rate 3 K/min; quoting of the arithmetic mean from three determinations
4. Particle size determination via laser diffraction with dry dispersal: Mastersizer 2000 with Scirocco 2000 dispersion unit (Malvern Instruments Ltd., UK), determinations at a counterpressure of 1, 2 and 3 bar; Fraunhofer evaluation; dispersant RI: 1.000, obscuration limits: 0.0-10.0%, tray type: general purpose, background time: 7500 msec, measurement time: 7500 msec, procedure in accordance with ISO 13320-1 and the information in the technical manual and specifications from the instrument manufacturer; quoted in % by vol
5. Particle size determination via laser diffraction with wet dispersal: Mastersizer 2000 with Hydro 2000S wet-dispersion unit (Malvern Instruments Ltd., UK); dispersion medium deionised water; dispersant RI: 1.330; pump speed: 2000 rpm; stirrer speed: 2000 rpm; ultrasonic duration: 1 sec; ultrasonic level: 100%; tray type: general purpose; background time: 7500 msec; measurement time: 7500 msec; obscuration limits: 10.0-20.0% I; procedure in accordance with ISO 13320-1 and in accordance with the information in the technical manual and specifications from the instrument manufacturer; quoted in % by vol.
6. Particle size determination by dry sieving via a sieve tower: Retsch AS 200 control, Retsch (Germany); amount of substance: about 110.00 g; sieving time: 30 minutes; amplitude intensity: 1 mm; interval: 5 seconds; analytical sieve with metal-wire fabric in accordance with DIN ISO 3310; mesh widths (in μm): 710, 600, 500, 400, 355, 300, 250, 200, 150, 100, 75, 50, 32; amount distribution per sieve fraction indicated in the tables as “% by weight of the sample weight”:
7. Iodometric determination of the content of ascorbic acid in the mixtures: the procedure consists of the steps titre determination of the iodine solution using sodium thiosulfate, checking by titration against an ascorbic acid standard substance of known content, titration of the carriers without ascorbic acid loading (blank value) and a 6-fold determination of the ascorbic acid content in the mixtures prepared, both before and after the mixing process, with subsequent calculations of the means and the standard deviations
   The basic procedure is also described in the specialist literature, such as, for example, in G. Jander, K. F. Jahr, H. Knoll “Maβanalyse—Theorie und Praxis der klassischen und der elektrochemischen Titrierverfahren” [Volumetric Analysis—Theory and Practice of Classical and Electrochemical Titration Methods], Verlag Walter de Gruyter, 1973 ISBN 3 11 005934 7
   The sample (sample weight depends on the ascorbic acid content in the mixture) is introduced into a 100 ml beaker and suspended with about 10 ml of demineralised water. The material is carefully dissolved with 25% sulfuric acid via a piston pipette with shaking, 1 ml of zinc iodide starch solution is subsequently added, and the mixture is immediately titrated with iodine solution until the colour changes from colourless to blue.

### Chemicals:

iodine solution 0.05 mol/l Merck KGaA (Germany) Art. No. 1.09099
zinc iodide starch solution Merck KGaA (Germany) Art. No. 1.05445
sulfuric acid 25% Merck KGaA (Germany) Art. No. 1.00716
micronised ascorbic acid obtained from ascorbic acid in a purity in accordance with Ph Eur, BP, JP, USP and E 300 (as described under materials)
ascorbic acid, prod. 83568.290, VWR (Germany); Ph Eur, NF, USP as ascorbic acid standard substance

### Equipment:

titroprocessor 682, Metrohm (Switzerland)
Dosimat 665, Metrohm (Switzerland)
20 ml brown-glass burette, Metrohm (Switzerland)
Ti stand 703 stirrer, Metrohm (Switzerland)
Research 5000 piston pipette, Eppendorf (Germany)
8. Spectrophotometric determination of the content of riboflavin in the mixtures: the procedure consists of the steps establishment of a calibration curve, checking by photometric measurement of a riboflavin standard substance of known content, photometric measurement of the carriers without riboflavin loading (blank value) and a 6-fold determinations of the riboflavin content in the mixtures prepared, both before and after the mixing process, with subsequent calculations of the means and the standard deviations
   The sample (sample weight depends on the riboflavin content in the mixture) is introduced into a 500 ml brown-glass volumetric flask, suspended with 5 ml of demineralised water, and 5 ml of 2M sodium hydroxide solution are then added. The suspension is shaken for 10 minutes, and 100 ml of demineralised water and 2.5 ml of glacial acetic acid are then added successively, the mixture is shaken again briefly and made up to the 500 ml mark with demineralised water. About 70 ml of this yellow suspension are centrifuged at 3800 rpm for 3 min. 20.0 ml of the supernatant are pipetted into a 200 ml brown-glass volumetric flask, 3.5 ml of 14 g/l sodium acetate solution (Art. No. 1.06268) are added, and the mixture is made up to 200 ml with demineralised water. This solution is measured against the solvent in the photometer at 444 nm and a cell thickness of 1 cm.

### Chemicals:

2M sodium hydroxide Merck KGaA (Germany) Art. No. 1.09136
glacial acetic acid Merck KGaA (Germany) Art. No. 1.00063
sodium acetate MerckKGaA (Germany) Art. No. 1.06268
micronised riboflavin obtained from riboflavin in a purity in accordance with Ph Eur, BP, USP and E 504 (as described under materials)
riboflavin Merck KGaA (Germany) Art. No. 500257, Ph Eur, BP, USP, E 101 as riboflavin standard substance

### Equipment:

Lambda 35 2-beam photometer Perkin Elmer (USA)
Plastibrand makro 2.5 ml disposable cells, Brand (Germany) Art. No. 759005
Heraeus Sepatech Minifuge T centrifuge (Germany) with 80 ml centrifuge tubes
Research 5000 piston pipette, Eppendorf (Germany)
20.0 ml glass volumetric pipette Hirschmann EM (Germany)
Blaubrand brown-glass volumetric flask, in accordance with ISO 1042, Brand (Germany)
   Directly Compressible DC Magnesium Hydroxide Carbonates Used and their Properties:

### Sample A:

Parteck Mg DC magnesium hydroxide carbonate heavy Ph Eur, BP, USP, E 504, Merck KGaA, Darmstadt (Germany), Art. No. 1.02440, batch: K0076840

### Sample B:

NutriMag MC DC magnesium carbonate heavy, pharm., gran. in purity BP, USP, Ph Eur; CALMAGS GmbH, Lüneburg (Germany); batch: 308075060

Additional characterisation of samples A and B with respect to bulk density, tapped density, BET surface area, BET pore volume, particle distribution via laser diffraction with wet dispersal (in water) and via tower sieving:

TABLE 1
Bulk density, tapped density, BET surface area, BET pore volume:
(Details on the measurement methods see under Methods)

| | | | BET surface | BET pore |
|---|---|---|---|---|
| | Bulk density | Tapped density | area | volume |
| Sample | (g/ml) | (g/ml) | (m²/g) | (cm³/g) |
| Sample A | 0.53 | 0.75 | 44.4 | 0.20 |
| Sample B | 0.63 | 0.77 | 11.5 | 0.08 |

TABLE 2
Particle distribution determined via laser diffraction with
wet dispersal in water:
FIGURES in μm (details on the measurement method
see under Methods)

| Sample | D(5) | D(10) | D(20) | D(25) | D(30) | D(50) | D(75) |
|---|---|---|---|---|---|---|---|
| Sample A | 2.26 | 5.62 | 11.87 | 14.10 | 16.11 | 23.76 | 36.09 |
| Sample B | 1.24 | 2.02 | 4.37 | 6.01 | 7.84 | 15.96 | 31.37 |

| Sample | D(90) | D(95) | D(99) | D(100) |
|---|---|---|---|---|
| Sample A | 50.07 | 59.41 | 75.81 | 93.54 |
| Sample B | 67.61 | 197.37 | 455.59 | 684.57 |

TABLE 3
Particle distribution determined via tower sieving:
FIGURES in % by weight (details on the measurement method see under Methods)

| Sample | <32 μm | 32-50 μm | 50-75 μm | 75-100 μm | 100-150 μm | 150-200 μm | 200-250 μm |
|---|---|---|---|---|---|---|---|
| Sample A | 12.2 | 53.3 | 27.3 | 6.1 | 0.2 | 0.2 | 0.1 |
| Sample B | 0.1 | 0.3 | 0.4 | 1.1 | 5.2 | 10.9 | 12.3 |

| Sample | 250-300 μm | 300-355 μm | 355-400 μm | 400-500 μm | 500-600 μm | 600-710 μm | >710 μm |
|---|---|---|---|---|---|---|---|
| Sample A | 0.1 | 0.1 | 0.1 | 0.0 | 0.1 | 0.2 | 0.0 |
| Sample B | 11.8 | 12.5 | 7.5 | 21.2 | 15.3 | 1.4 | 0.0 |

Micronised Model Active Ingredients Used and their Properties:

Model active ingredient micronised ascorbic acid: Grinding of a commercially available pulverulent ascorbic acid having a purity in accordance with Ph Eur, BP, JP, USP, E 300 on an Aeroplex model 200 AS spiral jet mill from Hosokawa Alpine, Augsburg (Germany) under nitrogen as protective gas; the target particle size D(50) measured by laser diffraction with dry dispersal is in the range from 4 μm to 6 μm
the more precise particle distribution of the material used is shown by the following table.

TABLE 4
Particle distribution of the micronised ascorbic acid determined via laser
diffraction with dry dispersal (various pressure conditions):
Figures in μm (details on the measurement method see under Methods)

| | Pressure | | | | |
|---|---|---|---|---|---|
| | 1 bar | | | | |
| Sample | D(10) | D(25) | D(50) | D(75) | D(90) |
| Micronised | 1.90 | 3.61 | 5.61 | 8.13 | 10.82 |
| ascorbic acid | | | | | |
| | Pressure | | | | |
| | 2 bar | | | | |
| Sample | D(10) | D(25) | D(50) | D(75) | D(90) |
| Micronised | 1.59 | 3.12 | 4.91 | 7.22 | 9.76 |
| ascorbic acid | | | | | |
| | Pressure | | | | |
| | 3 bar | | | | |
| Sample | D(10) | D(25) | D(50) | D(75) | D(90) |
| Micronised | 1.40 | 2.85 | 4.52 | 6.68 | 9.05 |
| ascorbic acid | | | | | |

### Model Active Ingredient Micronised Riboflavin:

Grinding of a commercially available pulverulent riboflavin having a purity in accordance with Ph Eur, BP, USP, E 504 on an Aeroplex model 200 AS spiral jet mill from Hosokawa Alpine, Augsburg (Germany) under nitrogen as protective gas; the target particle size D(50) measured by laser diffraction with dry dispersal is in the range from 1.5 μm to 2.5 μm
the more precise particle distribution of the material used is shown in the following table:

TABLE 5
Particle distribution of the micronised riboflavin determined via laser
diffraction with dry dispersal (various pressure conditions):
Figures in μm (details on the measurement method see under Methods)

| | Pressure | | | | |
|---|---|---|---|---|---|
| | 1 bar | | | | |
| Sample | D(10) | D(25) | D(50) | D(75) | D(90) |
| Micronised | 0.54 | 0.96 | 2.08 | 3.89 | 5.88 |
| riboflavin | | | | | |
| | Pressure | | | | |
| | 2 bar | | | | |
| Sample | D(10) | D(25) | D(50) | D(75) | D(90) |
| Micronised | 0.43 | 0.73 | 1.72 | 3.28 | 4.74 |
| riboflavin | | | | | |
| | Pressure | | | | |
| | 3 bar | | | | |
| Sample | D(10) | D(25) | D(50) | D(75) | D(90) |
| Micronised | 0.43 | 0.73 | 1.72 | 3.18 | 4.40 |
| riboflavin | | | | | |

### Example 1

### Determination of the Loading Capacity and Homogeneity of Various Amounts of Micronised Ascorbic Acid on Samples A and B after Mixing in a Tumble Mixer

### Principle:

In each case, mixtures comprising 2%, 5%, 7%, 10%, 20% and 30% of micronised ascorbic acid with the two DC magnesium hydroxide carbonate samples A and B were prepared
In order to establish the homogeneity of the mixtures, the ascorbic acid content were determined at 6 different points of these mixtures
The deviations of the relative standard deviations are indicators of the homogeneity of the mixtures and allow conclusions to be drawn on differences in the loading capacity

### Procedure:

In each case, the amounts of micronised ascorbic acid indicated in the table are added to the DC magnesium hydroxide carbonates samples A and B in a 250 ml wide-necked glass bottle (VWR Deutschland) and mixed in a laboratory tumble mixer (Turbula T2A, Willy A. Bachofen, Switzerland). After a mixing time of 15 minutes, the material is passed through a 1 mm sieve without mechanical loading, and any loose agglomerates present are carefully pressed through the sieve meshes using a sheet of paper. Mixing is subsequently continued for a further 15 minutes in the tumble mixer.

TABLE 6

| Micronised ascorbic | Amount of sample A or | Amount of micronised |
|---|---|---|
| acid (% by weight) | B (g) | ascorbic acid (g) |
| 2 | 98 | 2 |
| 5 | 95 | 5 |
| 7 | 93 | 7 |
| 10 | 90 | 10 |
| 20 | 80 | 20 |
| 30 | 70 | 30 |

After the mixing, the material is spread out on an area of 21×30 cm with the most uniform layer thickness possible, and samples are taken at 6 different points, their ascorbic acid content is determined, and the standard deviations are calculated.

### Result:

The theoretical amounts of ascorbic acid according to sample weight (in % by weight), the amounts of sample employed for the analytical determination of ascorbic acid (in g), the amounts of ascorbic acid actually found as arithmetic mean of 6 determinations (in % by weight) and the relative standard deviations S (rel) of these determinations (in %) are compared in the table

TABLE 7

| | | Amount of ascorbic | | | |
|---|---|---|---|---|---|
| Theoretical | | acid found as | | | |
| amount of | | arith. mean | | | |
| ascorbic acid | | [% by weight] | | S (rel) % | |
| [% by | Sample weight | Sample | Sample | Sample | Sample |
| weight] | [g] | A | B | A | B |
| 2 | 1.6749-2.3010 | 1.96 | 1.93 | 0.59 | 2.45 |
| 5 | 0.8463-1.1858 | 4.91 | 4.89 | 1.30 | 5.95 |
| 7 | 0.5336-0.7608 | 6.90 | 6.43 | 1.34 | 5.64 |
| 10 | 0.4251-0.5224 | 9.95 | 9.34 | 1.02 | 3.61 |
| 20 | 0.1697-0.3227 | 19.75 | 18.76 | 1.06 | 7.46 |
| 30 | 0.1019-0.2252 | 29.40 | 30.94 | 1.34 | 8.34 |

Sample A exhibits a lower relative standard deviation in the case of all mixtures than the samples prepared on the basis of sample B, i.e. the mixtures based on sample A have significantly better homogeneity.

### Example 2

### Comparative Investigation of the Adsorption Forces Between Micronised Ascorbic Acid and Samples A and B

### Principle:

In each case, mixtures of the two samples A and B with 1% of micronised ascorbic acid each were prepared, and their homogeneity was tested by determining the ascorbic acid contents at 6 different points of these mixtures.
These mixtures were subsequently mechanically loaded (in a tamping volumeter at 2500 and 20000 impacts and in a tower sieving machine), and the homogeneity of the mixtures was re-tested after this loading
The deviations of the relative standard deviations in the ascorbic acid content before and after mechanical loading are an indicator of the stability of the mixtures and thus also of the binding forces between the ascorbic acid particles and the carrier particles.

### Procedure:

148.5 g of sample A or sample B are weighed out into a 500 ml wide-necked glass bottle (VWR Deutschland) with 1.5 g of micronised ascorbic acid in each case and mixed in a laboratory tumble mixer (Turbula T2A, Willy A. Bachofen, Switzerland). After a mixing time of 15 minutes, the material is passed through a 1 mm sieve without mechanical loading, and any loose agglomerates present are carefully pressed through the sieve meshes using a sheet of paper. Mixing is subsequently continued for a further 15 minutes in the tumble mixer. After the mixing, the material is spread out on an area of 21×30 cm with the most uniform layer thickness possible, and samples are tested for their ascorbic acid contents at 6 different points, and the standard deviations are calculated

Each of these mixtures is subjected to a mechanical load:
a) A tamping load in a tamping volumeter, as described in Ph Eur 7th Edition (7.02. main part 2011 Volume 1 described under 2.9.34 Tapped density; the tamping volumeter shown on page 430 under FIG. 2.9.34-3 is used for powder samples having a defined fall height of 3+/−0.2 mm. In contrast to the number of tamping movements defined therein, the sample is subjected to 2500 impact movements. The material is subsequently carefully spread out on an area of 21×30 cm with the most uniform layer thickness possible, and samples are tested for their ascorbic acid contents at 6 different points, and the standard deviation is calculated.
b) As described under a); but with 20000 impact movements as load
c) A mechanical load in a model AS 200 control ‘g’ sieving tower from Retsch (Germany): to this end, the sample is spread out on the sieve tray (200 mm) and moved with an amplitude of 1.5 mm for 60 minutes (without interval). 6 samples are subsequently taken directly at various points of the sieve tray, the ascorbic acid content is determined, and the standard deviation is calculated.

### Result:

The tables show the amounts (sample weight) of sample employed for the analytical determination of ascorbic acid (in g), the amounts of ascorbic acid actually found as arithmetic mean of 6 determinations (in % by weight) and the relative standard deviations S (rel) of these determinations (in %). All figures are listed both before and also after mechanical loading.

TABLE 8
Content and S (rel) of ascorbic acid before and after
mechanical loading in the tamping volumeter after
2500 impacts

| | before mechanical loading | | | after mechanical loading | | |
|---|---|---|---|---|---|---|
| | | Ascorbic | | | Ascorbic | |
| | Sample | acid | S | Sample | acid | S |
| | weight | [% by | (rel) | weight | [% by | (rel) |
| | [g] | weight] | % | [g] | weight] | % |
| Sam- | 3.897-4.289 | 0.99 | 1.89 | 3.915-4.408 | 0.99 | 2.04 |
| ple A | | | | | | |
| Sam- | 3.570-4.147 | 1.07 | 1.91 | 3.568-4.364 | 1.06 | 4.91 |
| ple B | | | | | | |

TABLE 9
Content and S (rel) of ascorbic acid before and after
mechanical loading in the tamping volumeter after
20000 impacts

| | before mechanical loading | | | after mechanical loading | | |
|---|---|---|---|---|---|---|
| | | Ascorbic | | | Ascorbic | |
| | Sample | acid | S | Sample | acid | S |
| | weight | [% by | (rel) | weight | [% by | (rel) |
| | [g] | weight] | % | [g] | weight] | % |
| Sam- | 3.856-4.161 | 0.96 | 2.05 | 3.916-4.286 | 0.96 | 1.92 |
| ple A | | | | | | |
| Sam- | 3.915-4.588 | 1.06 | 2.11 | 3.758-4.347 | 1.07 | 3.73 |
| ple B | | | | | | |

TABLE 10
Content and S (rel) of ascorbic acid before and after
mechanical loading in a Retsch sieving tower

| | before mechanical loading | | | after mechanical loading | | |
|---|---|---|---|---|---|---|
| | | Ascorbic | | | Ascorbic | |
| | Sample | acid | S | Sample | acid | S |
| | weight | [% by | (rel) | weight | [% by | (rel) |
| | [g] | weight] | % | [g] | weight] | % |
| Sam- | 3.913-4.250 | 1.00 | 1.49 | 3.888-4.247 | 0.88 | 5.28 |
| ple A | | | | | | |
| Sam- | 3.860-4.158 | 1.08 | 3.66 | 3.926-4.205 | 1.23 | 76.70 |
| ple B | | | | | | |

Sample A exhibits a lower relative standard deviation in the case of all mixtures than the samples prepared on the basis of sample B, i.e. the mixtures based on sample A have a significantly lower separation tendency, also caused, inter alia, by stronger adsorption forces between the ascorbic acid particles and the carrier particles.

### Example 3

### Determination of the Loading Capacity and Homogeneity of Various Amounts of Micronised Riboflavin on Samples A and B after Mixing in a Tumble Mixer

### Principle:

In each case, mixtures comprising 5%, 10% and 20% of micronised riboflavin with the two DC magnesium hydroxide carbonate samples A and B were prepared
In order to establish the homogeneity of the mixtures, the riboflavin content were determined at 6 different points of these mixtures
The deviations of the relative standard deviations are indicators of the homogeneity of the mixtures and allow conclusions to be drawn on differences in the loading capacity

### Procedure:

The amounts of micronised riboflavin indicated in the table are in each case added to the DC magnesium hydroxide carbonates samples A and B in a 1000 ml plastic bottle (VWR Deutschland) and mixed in a laboratory tumble mixer (Turbula T2A, Willy A. Bachofen, Switzerland). After a mixing time of 1 minute, the material is passed through a 1 mm sieve without mechanical loading, and any loose agglomerates present are carefully pressed through the sieve meshes using a sheet of paper. Mixing is subsequently continued for a further 1 minute in the tumble mixer.

TABLE 10

| Micronised riboflavin | Amount of sample A or | Amount of micronised |
|---|---|---|
| [% by weight] | B [g] | riboflavin [g] |
| 5 | 285 | 15 |
| 10 | 270 | 30 |
| 20 | 240 | 60 |

After the mixing, the material is spread out on an area of 21×30 cm with the most uniform layer thickness possible, and samples are determined for their ascorbic acid content at 6 different points, and the standard deviations are calculated.

### Result:

The following are compared with one another in Table 11:
a) the theoretical amounts of riboflavin, according to sample weight in % by weight,
b) the amounts of samples in mg employed for the analytical determination of riboflavin,
c) the amounts of riboflavin actually found as arithmetic mean of 6 determinations in % by weight
   and
d) the relative standard deviations S (rel) of these determinations in %.

TABLE 11

| Theoretical | | Amount of riboflavin | | | |
|---|---|---|---|---|---|
| amount of | | found as arith. mean | | | |
| riboflavin | | [% by weight] | | S (rel) % | |
| [% by | Sample weight | Sample | Sample | Sample | Sample |
| weight] | [mg] | A | B | A | B |
| 5 | 246.32-293.30 | 4.94 | 4.44 | 1.53 | 1.60 |
| 10 | 129.14-138.54 | 9.87 | 9.36 | 1.62 | 5.20 |
| 20 | 61.34-90.46 | 19.51 | 19.67 | 0.59 | 6.67 |

Sample A exhibits a lower relative standard deviation in the case of all mixtures than the samples prepared on the basis of sample B, i.e. the mixtures based on sample A have significantly better homogeneity.

### Example 4

### Comparative Investigation of the Adsorption Forces Between Micronised Riboflavin and Samples A and B

### Principle:

In each case, mixtures of the two samples A and B with 5% and 10% of micronised riboflavin each were prepared, and their homogeneity was tested by determining the riboflavin contents at 6 different points of these mixtures.
These mixtures were subsequently mechanically loaded in a tower sieving machine, and the homogeneity of the mixtures was re-tested after this loading. The deviations of the relative standard deviations in the riboflavin content before and after mechanical loading are indicators of the stability of the mixtures and thus also of the bonding forces between the riboflavin particles and the carrier particles.

### Procedure:

The mixed riboflavin samples from Example 3 with a content of 5% and 10% are subjected to mechanical loading via a model AS 200 control ‘g’ tower sieving machine from Retsch (Germany) for 60 minutes. To this end, the samples are moved on the sieve tray (200 mm) with an amplitude of 1.5 mm without intervals. 6 samples are subsequently taken directly at various points of the sieve tray, the ascorbic acid content is determined, and the standard deviation is calculated.

### Result:

The tables show the amounts of sample (sample weight) employed for the analytical determination of riboflavin (in mg), the amounts of riboflavin actually found as arithmetic mean from 6 determinations in % by weight, and the relative standard deviations S (rel.) from these determinations in %. All figures are listed both before and also after mechanical loading.

TABLE 12
Content and S (rel) of riboflavin before and after
mechanical loading on a Retsch tower sieving machine;
nominal loading with 5% of riboflavin

| | before mechanical | | | | |
|---|---|---|---|---|---|
| | loading according | | | | |
| | to table . . . | | after mechanical loading | | |
| | Riboflavin | | | Riboflavin | |
| | [% by | S (rel) | Sample weight | [% by | S(rel) |
| | weight] | % | [g] | weight] | [%] |
| Sample A | 4.94 | 1.53 | 258.32-294.04 | 4.63 | 7.46 |
| Sample B | 4.44 | 1.60 | 260.20-292.50 | 3.88 | 23.70 |

TABLE 13
Content and S (rel) of riboflavin before and after mechanical loading
in a sieving tower; nominal loading with 10% of riboflavin

| | before mechanical | | | | |
|---|---|---|---|---|---|
| | loading according | | | | |
| | to table | | after mechanical loading | | |
| | Riboflavin | | | Riboflavin | |
| | [% by | S (rel) | Sample weight | [% by | S(rel) |
| | weight] | [%] | [g] | weight] | [%] |
| Sample A | 9.87 | 1.62 | 139.66-165.24 | 10.01 | 7.99 |
| Sample B | 9.36 | 5.20 | 149.20-239.10 | 7.37 | 11.36 |

Sample A exhibits a lower relative standard deviation in the case of all mixtures than the samples prepared on the basis of sample B, i.e. the mixtures based on sample A have a lower separation tendency, also caused, inter alia, by strong adsorption forces between the riboflavin particles and the carrier particles.

1. Solid formulation, characterised in that it comprises
a) at least one porous carrier consisting of magnesium hydroxide carbonate, and
b) one or more functional substances.

2. Formulation according to claim 1, characterised in that it comprises an ordered mixture consisting of 50 to 99.9% by weight of magnesium hydroxide carbonate and 50 to 0.1% by weight of at least one micronised functional component.

3. Formulation according to claim 1, characterised in that the magnesium hydroxide carbonate is a material having a BET surface area in the range from 25 to 70 m²/g, preferably greater than 44 m²/g, particularly preferably greater than 50 m²/g, and a bulk density in the range from 0.40 to 0.60 g/ml and a tapped density in the range from 0.50 to 0.80 g/ml.

4. Formulation according to claim 1, characterised in that it comprises a directly compressible magnesium hydroxide carbonate having a particle diameter (laser, D₅₀) in the range between 10 and 60 μm.

5. Formulation according to claim 1, characterised in that it comprises at least one functional component in the form of a micronised substance having a particle size (laser, D₅₀) of 1 to 20 μm, preferably of 1 to 10 μm.

6. Formulation according to claim 1, characterised in that the porous magnesium hydroxide carbonate present as carrier form, together with one or more functional substances, a stable ordered mixture having particularly good homogeneity.

7. Formulation according to claim 5, characterised in that it comprises a functional component from the area of pharmaceutical active ingredients, diagnostic agents, food supplements, cosmetics, herbicides, fungicides, reagents, dyes, dietary minerals, catalysts or enzymes or microorganisms.

8. Formulation according to claim 1, characterised in that it is a mixture which is distinguished by pronounced homogeneity and stability of the mixture, even under mechanical load.

9. Formulation according to claim 8, characterised in that it comprises active ingredients and assistants selected from the group flow improvers, binders, lubricants, sweeteners and, polymers.

10. Formulation according to claim 1, characterised in that it is a powder or tablet.

11. Formulation according to claim 1, characterised in that it is a powder or tablet in which the pharmaceutical active ingredient is present in a low dose.

12. A mixture in solid form containing a formulation according to claim 1.

13. An active ingredient-containing tablet, capsule, powder, ointment, cream, suspension, or dispersion containing a formulation according to claim 1.

14. A pharmaceutical composition for oral or dermal administration containing a formulation according to claim 1.

15. A pharmaceutical, cosmetic, agricultural or industrial composition containing a formulation according to claim 1.

16. A food supplement containing a formulation according to claim 1.

17. Process for the preparation of a formulation according to claim 1, characterised in that at least one porous carrier, consisting of a porous magnesium hydroxide carbonate having a large surface area, with at least one functional substance in the form of a micronised powder are mixed intensively with one another in a mixer selected from the group tumble mixers, screw cone mixers, compulsory mixers, stirred mixers, high-speed mixers and fluidised-bed mixers.
